(19) United States
(12) Patent Application Publication
(10) Pub. No.: US 2019/0030335 A1
(43) Pub. Date: Jan. 31, 2019

(54) REGULATION OF THALAMIC ACTIVITY FOR CONTROL OF AGGRESSION

(71) Applicant: Joel Steven Goldberg, Hillsborough, NC (US)

(72) Inventor: Joel Steven Goldberg, Hillsborough, NC (US)

(21) Appl. No.: 16/139,062

(22) Filed: Sep. 23, 2018

(51) Int. Cl. A61N 1/36; A61N 1/04

(52) U.S. Cl. CPC A61N 1/36025; A61N 1/0529; A61N 1/0456; A61N 1/36017

(57) ABSTRACT

War of aggression, the root cause of which exists in the mind, produces suffering to constituents and their leaders. Multiple sites in the human brain are associated with aggression; however, a universal site responsible for all aggressive actions has not been found. Electrical stimulation of the brain has changed aggressive behavior, and electrical stimulation in some instances can overcome voluntary behavior. Of the many methods to stimulate the brain remotely and modify aggressive behavior, stimulation of the senses of touch, sight and/or sound is advantageous since it bypasses the blood brain barrier and the electrical impedance of soft tissues and bone overlying the brain. Stimulation of the senses produces waves comprised of action potentials in the thalamus that can interfere or entrain existing action potential waves. The waves from stimulation have the potential to discriminate existing electrical activity of sites in the brain associated with aggressive behavior.

### CROSS-REFERENCES TO RELATED APPLICATIONS

None

### FEDERALLY FUNDED RESEARCH

None

### BACKGROUND OF THE INVENTION

Wars of aggression trace the history of civilization, but war of aggression is not inevitable. According to archeologic findings, war has been a part of man's culture prior to the development of agriculture. Remains of mass burials associated with weapons and inflicted destruction of human bones are the best evidence that hunter-gatherer societies were involved with aggressive warfare.

The establishment of sovereign nations by the Peace of Westphalia in 1648 did not predetermine nations to war nor did it predetermine a single global nation as a prerequisite for world peace. By themselves, sovereign states define the geographic, economic, political, and cultural boundaries among countries, but existence of the sovereign state does not promote war of aggression. The root cause of all wars of aggression is within the minds of those who inhabit warring countries, primarily their leaders and their supporting constituents.

War of Aggression is Defined

Unlike emotions of hate, fear, and anger, aggression is an action. This action, whether impulsive or premeditative, is:
1. Making attacks or encroachments, especially the unprovoked violation by one country of the territorial integrity of another
2. A forceful action (such as an unprovoked attack), especially with the intent to dominate or master

Humans, along with other primates, have the innate ability to aggress. In many ways aggression is responsible for natural selection, and in some ways aggression has improved mankind by fostering competitions with positive outcomes. Many developments in science and the arts came to fruition because of this innate aggression. Aggression is partially responsible for man's dominance as a species. Overcrowding from population growth with limitation of resources may increase aggression. Aggression of war is a consequence of intense conflict among nations that cannot be resolved by negotiation. Such aggression in the minds of political leaders who are psychopaths, megalomaniacs, or narcissists has led to cruelty, death, and destruction of infrastructure.

Some of the causes of conflict among nations that produce aggression in the minds of leaders and their constituents:

Culture

The culture within a sovereign state influences what one considers right and wrong. Stealing, cheating, lying, and slavery are condoned by different cultures depending upon circumstances. Religious, racial, or economic discrimination is widely practiced and socially accepted in some societies. Even homicide and genocide become morally acceptable when the influences of the culture are so strong they produce acceptable ideologies of hatred.

In addition, what has been considered morally correct during a time period in one culture may not be morally correct at a later time period in that culture. Culturally accepted by the masses, slavery was not considered immoral for thousands of years. Present day, culturally accepted professional boxers who fight until a party is incapacitated for the win are only dissimilar in degree of injury to culturally accepted gladiators who fought until death during the Roman period. In summary, culture has a great influence on what one perceives to be right and wrong. Exactly how culture changes executive functions particularly of the prefrontal cortex is not known, but epigenetic influence may be a plausible explanation.

Ascribing Anthropomorphic Values to the Sovereign State

There are approximately 195 sovereign nations on earth that define geographic, economic, political, and cultural boundaries. Most of these sovereign states have attributes that humanize their existence. When nations take on human characteristics, national thought can be filled with deleterious emotions. Attributes ascribed to countries such as strong or weak, aggressive or docile, liberal or conservative, and rich or poor humanize the sovereign state. Military strength becomes synonymous with a strong nation that can defend itself and become an aggressor.

Retribution for Past Events

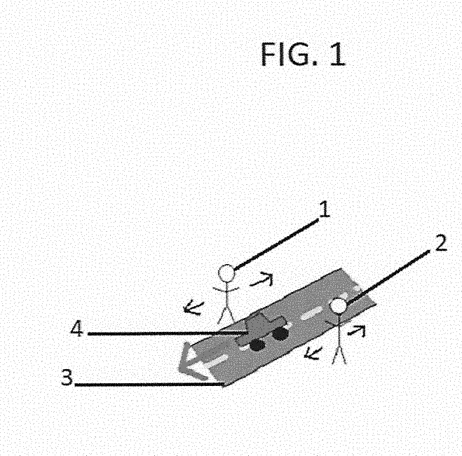

Humans have an innate desire to reconcile the past, especially when they perceive that they have been the subject of wrongdoing. Retribution rarely leads to reconciliation. It is counterintuitive, but special relativity teaches that events in time and space are variants; that is, they change depending on frame of reference, and subsequently, past events may not be perceived the same by different observers. For example; FIG. 1 shows a car driving down a road with two observers, one on each side of the road. Observer 1 will state that the car is traveling from left to right, and Observer 2 will state that the car is traveling from right to left, even though both observers are in stationary frames of reference relative to the road. (FIG. 1)

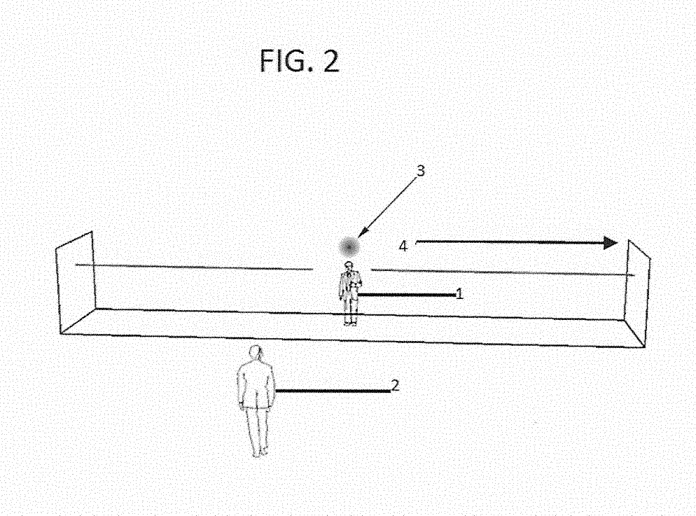

Simultaneity of an event is also relative to the frame of reference. In FIG. 2, an example of one of Albert Einstein's thought experiments, a light beam originates at the center of a moving train car with Observer 1 also in the center of the car. Observer 1 will record the light hitting the front and back of the car at the same time. However, Observer 2 who is standing on a platform will record the light first hitting the rear of the car and then the front of the car. Observer 2 will observe the distance traveled by the rear beam to be less than the distance traveled by the front beam. Since the speed of light is a constant, to Observer 2 the time for the light to reach the rear of the car is less than the time to reach the front of the car. (FIG. 2)

An event in time and space may not be simultaneous for two observers in different frames of reference. In fact, any two or more observers occupy slightly different space-time coordinates and their perceptions of an event may be slightly different. As is well known, eyewitness accounts of an event can differ for different observers. Therefore, retribution for past events may be difficult without a common perception of what was the wrongdoing. In summary, the past is immutable, but the perception of an event by multiple observers can be different. This slight difference in perception could hinder peaceful negotiations, since brain activity is often chaotic where small changes in the initial condition may lead to significantly different future perceptions and interpretations.

Territoriality and Scarce Resources

Disputed geographic borders among sovereign states is a frequent cause of conflict. Moreover, scarce resources, some of which may be acquired through free trade, can produce conflict when war of aggression is the most expedient method to steal resources.

Religious Claims

Religion is a powerful force that unites and separates man. War of aggression in the name of a religion or religious belief is based on faith. Numerous examples exist in history, including the Crusades and recent wars in Bosnia, Middle East, Ireland, and Somalia.

Ideologies

Similar to religious claims, ideologies may not be founded in moral reasoning but rather become dogma and difficult to disprove as a cause of war of aggression.

Mutual Assured Destruction does not Exist

When the balance of power among nations becomes an imbalance of power, the minds of leaders and their constituents of more powerful warring nations perceive an opportunity to increase the strength of their nations often by war of aggression.

These are some of the reasons for conflict that can lead to war of aggression between sovereign states.

Technologic Advances Outpace Moral Development

In the last 150 years, technological developments from basic science discoveries such as the relationship between energy and matter, structure and function of DNA, and chemical synthesis has led to nuclear bombs, genetic engineering of virulent organisms, and potential for chemical warfare. Each of these developments has the capacity to cripple or destroy civilization permanently as we know it on earth. Whether we have been lucky that the living planet has not been destroyed or moral development has grown enough to keep us from self-destruction, one cannot say for sure, but the extrapolation of rapid growth of lethal technologies has the capacity to destroy civilization, especially when leaders and their constituents behave with impulsive aggression.

Peace Psychology has Helped but not Eliminated Serious Aggression

In the last fifty years much has been written about peace psychology. Resolution of conflicts among nations can be understood according to game theory. Win-lose (competitive), lose-lose (compromise), or win-win (cooperative) are outcomes of conflict negotiations. Win-win outcomes are associated with sustainable peace, because all parties gain and further negotiation is unlikely to achieve a better outcome even when there is full disclosure. Unfortunately, advances in peace psychology have not significantly reduced the number of conflicts that lead to wars of aggression.

World Leadership and Wars of Aggression

World leaders who control arsenals of weapons, particularly nuclear, biologic and chemical, who have megalomaniacal personalities and constituents who follow these leaders are the most serious concern for disruption of world order. These megalomaniacal leaders, who in the past were psychopaths, inflicted severe harm to their constituents and the constituents of other nations and have a disregard for the feelings of others. Some of these leaders have had such hatred in their hearts that defeat of the enemy included inflicting cruelty and pain in excess of that required for the win. Other pathologic personalities of leaders who are charismatic and dictatorial can influence their constituents and promote aggression.

Neurophysiology of Aggression

A complex behavior such as aggression has not been proven to be caused by distinct brain structures and functions in all humans. Although not completely elucidated, aggression involves activity within the thalamus, amygdala, prefrontal cortex, hypothalamus, and caudate nucleus.

TABLE 1
Commonly accepted structures associated with aggressive activity

| Location | Activity |
|---|---|
| Thalamus | Excitatory and inhibitory |
| Amygdala | Excitatory |
| Prefrontal cortex | Inhibitory |
| Hypothalamus | Excitatory |
| Caudate nucleus | Inhibitory |

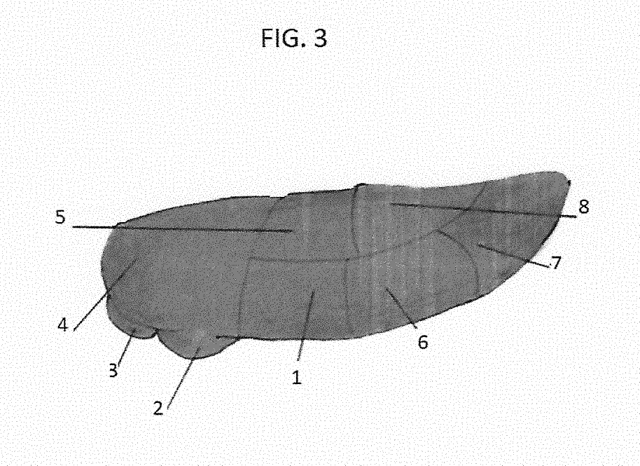

The thalamus receives afferent input from the senses. Tactile sensation is relayed in the ventral posterior medial and ventral lateral thalamus. Visual input is relayed in the lateral geniculate bodies, and auditory input is relayed in the medial geniculate bodies. (FIG. 3)

Located within the temporal lobe, the amygdala is a composite structure of thirteen nuclei that has been associated with emotions and aggression. (Coccaro, McCloskey, Fitzgerald, & Phan, 2007) The lateral nucleus of the amygdala receives sensory input from the thalamus, and the central nucleus of the amygdala is an output region for emotional responses. In vertebrae evolution, the amygdala is an old structure as is the action of aggression. Since the amygdala receives direct input via the thalamus, its activity is sensory dependent.

The main areas of the prefrontal cortex associated with aggression are the prefrontal orbital and prefrontal medial cortex. The “top down” inhibitory regulation of amygdala activity by these cortices in humans has supporting evidence and may have occurred later in evolution separating man from lower primates. (Siever, 2008) We know from human studies of individuals who have suffered lesions or electrical stimulation of the prefrontal cortex that aggression can be amplified or attenuated. (Choy, Raine, & Hamilton, 2018; Dambacher et al., 2015)

The hypothalamus receives direct input from the thalamus and regulates the neuroendocrine response to aggression. (Hashikawa, Hashikawa, Falkner, & Lin, 2017)

The caudate nucleus can be reliably stimulated in primates to inhibit aggression. (Delgado, 1969)

Brainwave Entrainment and Brainwave Interference Defined

Brainwave entrainment is a phenomenon where normal brain electrical activity aligns with an external stimulus. Entrainment was first observed by Christiaan Huygens in 1666 who observed synchrony of two pendulum clocks mounted next to each other on the same support. Entrainment occurs in coupled periodic and chaotic systems, both of which are found in brain electrical activity.

Brainwave interference is an activity where two or more brainwaves change amplitude and/or frequency according to the supposition of said brainwaves with one or more waves generated from outside the nervous system.

Evidence that External Forces could Produce Changes in the Brain Activity and Behavior

Drugs that cross the blood brain barrier can produce changes in the mind that are indistinguishable from psychosis. Numerous world leaders have been suspect of being under the influence of alcohol, opioids, and stimulants while they made important international decisions. For example, many Nazi leaders were under continued influence of amphetamines. Even if a “peace drug” were discovered, leaders and their constituents may not consent to self-administration. Some drugs such as opioids, cannabinoids, ethanol, and serotonin uptake inhibitors are reported in some individuals to produce peaceful states of mind, but it is doubtful that administration of these drugs will curtail human aggression.

Dr. José Delgado showed that direct stimulation of the brain with implanted electrodes can produce complex behaviors, and that the intensity of the stimulus can override volition or normal behaviors. (Delgado, 1969)

The laws of electricity and magnetism predict that ionizing electromagnetic radiation should change the electrical activity of the brain and behavior. Energy could be transferred to the brain by electric fields, magnetic fields, or electromagnetic radiation. Penetrating and targeting electromagnetic radiation into the brain would be very difficult with present technologies.

The second way electromagnetic radiation could influence the nervous system is through wave interference. Electromagnetic waves or sound waves at certain amplitudes, frequencies, and conformations should have influences on the activity of the brain and the mind. For example, in nature, some migrating birds are believed to be guided by the geomagnetic forces of the earth. Although dualism as described by Descartes was probably wrong, he may not have been that far off, since at the macro level electric and magnetic fields which are likely components of the mind, are a different substance than brain matter. Again, targeting electromagnetic waves and producing wave interference at specific locations within the brain would be very difficult with existing technologies.

Lastly, external energy can be transmitted to the brain through known sensory pathways, which is the focus of this invention. Touch, sight, and sound can be activated by external forces and the information is transmitted to various regions of the brain as action potential waves. The advantages of such a system would be that the impedance of the skull and soft tissues would be circumvented. Furthermore, wave interference and entrainment are more likely when similar waves and oscillations occur in a common medium.

Partial Coherence of Hemispheric Brain Activity May Decrease Aggression

Of the many mind states that have been studied, including those induced by drugs and electromagnetic energy, partial coherence between hemispheres is often associated with tranquility within the mind. This tranquility can be produced during meditation where thoughts can produce partial hemispheric coherence. A paranormal effect from transcendental meditation claims that 1% of individuals practicing transcendental meditation in a given area could encourage the well-being within the local environment by decreasing crime, terrorist activity, and warfare.

### DETAILED DESCRIPTIONS OF THE DRAWINGS

FIG. 1 shows two observers. Label 1 is observer 1, Label 2 is observer 2. Label 3 is a road. Label 4 is a car.

FIG. 2 shows two observers and a light source. Label 1 is an observer who sees light traveling from a single source an equal distance. Label 2 is an observer who sees light traveling an unequal distance from a single source. Label 3 is a light source. Label 4 is the direction of motion.

FIG. 3 shows the topographical organization of the thalamus with medial and lateral geniculate bodies. Label 1 is the ventral posterior medial and ventral lateral thalamus. Label 2 is the lateral geniculate body. Label 3 is the medial geniculate body. Label 4 is the pulvinar. Label 5 is the ventral posterior nucleus. Label 6 is the ventral lateral nucleus. Label 7 is the ventral anterior nucleus. Label 8 is the lateral dorsal nucleus.

### DETAILED DESCRIPTION OF THE INVENTION

Proposition #1

Human aggression can be changed by electrical stimulation of the brain, and some of these behavioral changes can overcome aggressive behaviors of volition.

Proposition #2

Aggressive behavior may be modified by stimulation of human senses, particularly the tactile, visual, and auditory senses.

Proposition #3

Modification of aggressive behavior by stimulation of the senses occurs in the thalamus through entrainment and/or interference and then progresses to other sites in the brain.

Remote Neurophysiologic Methods to Control Aggressive Behavior

Although ionizing and non-ionizing remote electromagnetic radiation have been extensively studied to control human behavior, the results are not reproducible, probably because penetration and targeting such radiation to specific areas of the brain are beyond present technologies.

Technically, the easier method to transfer energy to the brain would be to use the existing sensory pathways to produce changes in action potentials that could affect local areas of the brain through interference and/or entrainment. The stimulation will travel directly to the thalamus where modulation can occur. Connectomes from the thalamus project to all the suspected locations that may be responsible for aggression, including amygdala, prefrontal cortex, hypothalamus and caudate nucleus. Since the electrical activity of various areas of the brain are different, the external induced changes in action potentials to different sites within the brain could be discriminated. Using the existing sensory system bypasses the problems associated with the blood brain barrier, electrical impedance from the skin, subcutaneous tissue, and bone insulating the brain, and heat produced by ionizing radiation.

Wave interference and entrainment is most likely to occur in waves of the same character. For example, sound waves interfere or entrain sound waves, light waves interfere or entrain electromagnetic waves, and waves from action potentials interfere or entrain other waves from action potentials. Recordings from the thalamus would enable one to modulate electrical activity to other areas of aggression and design stimuli that would interfere or entrain with existing pathways.

### Benefits of to Society

Wars of aggression bring about the lowest forms of human behavior. War hurts people, ruins lives, and destroys infrastructure. Irreconcilable conflict usually precedes war, and encouraging peace by taming aggression in world leaders and their constituents should be a primary goal of humanity. Peace with mutually assured destruction (MAD) has kept us from a world war since 1945. However, MAD may not be sustainable because technologic advances in warfare come at a rapid pace. Worldwide defense expenditures in 2015 exceeded a trillion US dollars and are projected to rise. If this money could be allocated to problems of poverty, health, education, and infrastructure, the world would benefit. When there is cooperation and abundant resources, many projects such as the Manhattan project, human genome project, Great Wall of China, pyramids of Egypt, Hoover Dam, and the eradication of polio and smallpox viruses can be accomplished. The human mind has an uncanny ability to solve the most difficult of problems. In this era of rapid weapon development, discovering the details of taming human aggression by interfering or entraining thalamic activity through the use of targeted stimuli to the senses is vitally important.

### REFERENCES

Choy, O., Raine, A., & Hamilton, R. H. (2018). Stimulation of the Prefrontal Cortex Reduces Intentions to Commit Aggression: A Randomized, Double-Blind, Placebo-Controlled, Stratified, Parallel-Group Trial. J Neurosci, 38(29), 6505-6512. doi: 10.1523/JNEUROSCI.3317-17.2018
Coccaro, E. F., McCloskey, M. S., Fitzgerald, D. A., & Phan, K. L. (2007). Amygdala and orbitofrontal reactivity to social threat in individuals with impulsive aggression. Biol Psychiatry, 62(2), 168-178. doi: 10.1016/j.biopsych.2006.08.024
Dambacher, F., Schuhmann, T., Lobbestael, J., Arntz, A., Brugman, S., & Sack, A. T. (2015). Reducing proactive aggression through non-invasive brain stimulation. Soc Cogn Affect Neurosci, 10(10), 1303-1309. doi: 10.1093/scan/nsv018
Delgado, José Manuel Rodriguez. (1969). Physical control of the mind; toward a psychocivilized society (1st ed.). New York: Harper & Row.
Hashikawa, Y., Hashikawa, K., Falkner, A. L., & Lin, D. (2017). Ventromedial Hypothalamus and the Generation of Aggression. Front Syst Neurosci, 11, 94. doi: 10.3389/fnsys.2017.00094
Siever, L. J. (2008). Neurobiology of aggression and violence. Am J Psychiatry, 165(4), 429-442. doi: 10.1176/appi.ajp.2008.07111774

Having described my invention, I claim:

1. A method to decrease aggressive behavior in a human by remote stimulation of the senses in said human, causing interference of waves of thalamic action potentials.

2. The method of claim 1 where the sense is touch.

3. The method of claim 1 where the sense is sight.

4. The method of claim 1 where the sense is sound.

5. A method to decrease aggressive behavior in a human by remote stimulation of the senses in said human, causing entrainment of waves of thalamic action potentials.

6. The method of claim 2 where the sense is touch.

7. The method of claim 2 where the sense is sight.

8. The method of claim 2 where the sense is sound.
